(19) United States
(12) Patent Application Publication
(10) Pub. No.: US 2024/0397979 A1
(43) Pub. Date: Dec. 5, 2024

(54) METHODS FOR DELIVERING BENEFICIAL NUTRIENTS TO INVERTEBRATES THROUGH A MICROORGANISM DEFICIENT IN ANTINUTRIENTS

(71) Applicant: APIX BIOSCIENCES, Wingene (BE)

(72) Inventors: Thierry BOGAERT, Wingene (BE); Sharoni SHAFIR, Wingene (BE); Geraldine WRIGHT, Oxford (GB)

(21) Appl. No.: 18/693,692

(22) PCT Filed: Sep. 27, 2022
(86) PCT No.: PCT/EP2022/076791

§ 371 Date: Mar. 20, 2024

(30) Foreign Application Priority Data
Sep. 28, 2021 (BE) BE2021/5758
Oct. 4, 2021 (BE) BE2021/5775

(51) Int. Cl. A23K 50/90; A01K 53/00; A23K 10/18; C12N 1/16; C12N 15/90

(52) U.S. Cl. CPC A23K 50/90; A01K 53/00; A23K 10/18; C12N 1/16; C12N 15/905

(57) ABSTRACT

Provided is a method for feeding invertebrates, comprising ▪ Providing a pollen substitute or supplement composition for invertebrates, in particular for honey bees and bumble bees out of yeast selected from the group consisting of S. cerevisiae, Xanthophyllomyces sp., S. japonicus or Yarrowia lipolytica, ▪ Administering the composition to invertebrates, wherein the composition is essentially free of ergosterol or zymosterol or combinations thereof.

### BACKGROUND

Honey bees are very important in modern agriculture for the pollination of crops and for the production of honey, royal jelly, and wax. Honeybee colonies face a myriad of problems posed by their dense populations, management practices, and agriculture. In particular, modern agriculture in crops where bees are used for pollination causes them to be exposed to pesticides. Keeping them in high densities where there is little forage means that they inevitably face malnutrition that leads to increased prevalence of pathogens and parasites.

Beekeepers have historically allowed bees to feed themselves. Honey bees forage for nectar and pollen on flowering plants. Anthropogenic changes to landscape management have inevitably resulted in substantially fewer flowering plants, especially in agricultural environments. Because of these changes, forage is limited in availability and scale. Trying to replace natural forage that bees collect locally with pollen collected by other colonies in rich locations is costly, pollen is hard to keep fresh and pollen collected in the wild can carry pests, diseases, and pesticides.

As a result within the past 40 years, beekeepers have started to rely more on commercially produced or home-produced feeds composed of soy and yeast to replace pollen when landscapes do not support sufficient diversity and abundance of flowering plants to support honeybee colonies. As a result of recommendations made by the USDA as early as the 1950's, beekeepers began to feed honey bees with Brewer's yeast (S. cerevisiae) mixed with sugar. Yeast contains proteins, amino acids, vitamins and minerals. It contains <2% fat. Among its lipid compounds, the primary sterol of Brewer's yeast is ergosterol. Most commercial products sold as foods for honey bees contain Brewer's yeast, and some are made exclusively of Brewer's yeast (Table 1).

Yeasts are used to feed invertebrates, in particular honey bees or bumble bees. However, yeasts produce ergosterol. Ergosterol is an antinutrient, which decreases the performance, in particular the brood production of invertebrates, such as honey bees or bumble bees.

Moreover, cultivating yeasts requires the addition of sterols to the yeast culture. In this respect, WO 2021/133171 to the University of Delft discloses recombinant yeast cells expressing a protein having squalene-tetrahymanol cyclase activity or a protein having squalene-hopene cyclase activity. The recombinant yeast cells require less or no addition of sterols to the culture medium and produces a molecule that can functionally replace sterol function in the cell.

US2019/0090507 to Apix Biosciences stresses the importance of plant sterols and, in particular, of 24-methylene cholesterol, campesterol, β-sitosterol, and cholesterol to honey bees as essential nutrients. However, US2019/0090507 does not disclose ergosterol or zymosterol or a mixture thereof as a bee anti-nutrient.

### SHORT DESCRIPTION OF THE INVENTION

Accordingly, a first aspect of the invention is a method for feeding invertebrates comprising:
providing a non-pollen composition comprising a nutritionally effective amount of ergosterol-free yeast; or
providing a non-pollen composition comprising a nutritionally effective amount of zymosterol-free yeast; and
administering the non-pollen composition to invertebrates.

Another aspect is a method for feeding invertebrates comprising:
providing a composition comprising yeast or parts, extracts, oils, mixtures or refinements thereof; and
administering the composition to invertebrates,
wherein the yeast is selected from the group consisting of Saccharomyces cerevisiae, Xanthophyllomyces sp., S. japonicus or Yarrowia lipolytica;
wherein yeast is modified, selected or enhanced not to express any ergosterol or zymosterol, and
wherein the yeast is optionally further genetically modified or selected to produce an invertebrate nutrient selected from the group consisting of cholesterol, desmosterol, 24-methylene cholesterol, and isofucosterol,

In another aspect, the yeast has been modified or enhanced as compared to a parent yeast to express a squalene-hopene cyclase enzyme or a squalene-tetrahymanol cyclase enzyme to enable growth under anoxic conditions and functioning of the cell without sterol in the membranes.

In another aspect, the invertebrate nutrient is produced under aerobic conditions.

In another aspect, the composition is a whole invertebrate diet, part of an invertebrate diet or a dietary supplement.

In another aspect, the composition is administered to invertebrates of the Apis or Bombus genera, in particular honey bees or bumble bees.

In another aspect, the composition is administered
in solid form such as a patty or in liquid form such as a solution or spray or powder or topical application,
inside or outside the hive.

In another aspect, the source of the ergosterol/zymosterol-free yeast or a mixture thereof is a non-pollen source selected from the group consisting of:
a fungus, in particular an extract, an oil or a refinement of Saccharomyces cerevisiae, or Yarrowia lipolytica and including but not limited to the following genera: Aspergillus, Botrytis, Candida, Cercospora, Cryptococcus, Fusarium (Gibberella), Hansenula, Kluyveromyces, Metschnikowia, Neurospora, Penicillium, Pichia, Puccinia, Rhodotorula, Saccharomyces, Schizosaccharomyces, Sclerotium, Torula, Torulopsis, Trichoderma, and Xanthophyllomyces (Phaffia), Zygosaccharomyces.

In another aspect, the ergosterol/zymosterol-free yeast or a mixture thereof is provided as part of a pollen substitute composition, wherein the pollen substitute composition comprises:
proteins in an amount from 10 w % to 50 w %, preferably of 20 w % to 40 w %,
fatty acids in an amount from 1 w % to 20 w %, preferably of 2 w % to 12 w %,
carbohydrates in an amount from 30 w % to 90 w %, preferably of 5 w % to 50 w %,
optionally vitamins, and
optionally minerals,
   wherein the total amount of components and optionally further components add up to 100 w % and wherein the w % are related to the total dry weight of the composition.

In another aspect, the ergosterol/zymosterol-free yeast or a mixture thereof is mixed with sucrose and/or invert syrup in an embodiment of 1-60% yeast, but preferably 10-50% yeast, and more preferably 15-30% yeast.

In another aspect, the composition further comprises cholesterol, desmosterol, 24-methylene cholesterol, and isofucosterol in an amount from 0.01-5% of the diet, preferably from 0.01-1% and even more preferably from 0.03-0.5% for honey bees and 0.03-0.8% for bumble bees as a percentage of the total (dry) weight of the pollen substitute composition.

### SHORT DESCRIPTION OF THE DRAWINGS

FIG. 1 shows the choice of cohorts of bees of diet with or without yeast free of ergosterol

FIG. 2 shows the survival of cohorts of bees confined to feed on diets containing specific concentrations of yeast containing ergosterol

FIG. 3 shows the threshold for the influence of yeast with ergosterol on the production of brood in bee diet per unit protein

FIG. 4 shows the increase in the length of time that a colony can produce brood when sterols required by bees are added to diet

FIG. 5 shows the survival of cohorts of bees confined to feed on diets containing ergosterol and inoculated with the gut pathogen, Nosema ceranae

### DETAILED DESCRIPTION OF THE INVENTION

The inventors have surprisingly found that cells or extracts of Saccharomyces cerevisiae, Xanthophyllomyces sp., S. japonicus or Yarrowia lipolytica may be administered to invertebrates, in particular to honey bees and to bumble bees, to deliver nutrients whilst avoiding antinutrients such as ergosterol or zymosterol. S. japonicus grows without oxygen (sterol production from squalene requires oxygen molecules) and naturally produces compound(s) capable of functionally replacing sterol function in S. japonicus (as shown by the team of Pronk, see WO 2021/133171 to the University of Delft). Such ergosterol deficient S. japonicus have a superior nutrient profile over S. japonicus grown in the presence of oxygen or other baker's yeasts that naturally contain ergosterol or other. Consequently, in one embodiment, S. japonicus can replace the bakers or other ergosterol containing yeast currently used in bee nutrition.

In another embodiment, the yeast cells have been enhanced or modified to express or produce an invertebrate nutrient selected from the group consisting of cholesterol, desmosterol, 24-methylene cholesterol, and isofucosterol. In a preferred embodiment, the enhanced or modified yeast is essentially ergosterol-free. That means that the yeast is not expressing antinutrients such as ergosterol or zymosterol in nutritionally effective amounts. In another preferred embodiment, the yeast has been modified to further express a squalene-hopene cyclase enzyme or a squalene-tetrahymenol cyclase enzyme to enable production of a molecule that can replace or complement the ergosterol function in yeast growth (as the sterol pathway is used to make other sterols than ergosterol) and enables growth under anoxic conditions.

Yeasts Preferred microorganisms are yeasts, preferably selected from the group consisting of Saccharomycetaceae, such as Saccharomyces cerevisiae, Saccharomyces pastorianus, Saccharomyces beticus, Saccharomyces fermentati, Saccharomyces paradoxus, Saccharomyces uvarum and Saccharomyces bayanus; Torulaspora, such as Torulaspora delbrueckii; Kluyveromyces, such as Kluyveromyces marxianus and Kluyveromyces lactis; Pichia, such as Pichia stipitis (also known as Scheffersomyces stipitis), Pichia pastoris; Ogataea, such as Ogataea parapolymorpha; Zygosaccharomyces, such as Zygosaccharomyces bailii; Brettanomyces, such as Brettanomyces intermedius, Brettanomyces bruxellensis, Brettanomyces anomalus, Brettanomyces custersianus, Brettanomyces naardenensis, Brettanomyces nanus, Dekkera bruxellis and Dekkera anomala; Metschnikowia; Issatchenkia, such as Issatchenkia orientalis; and Kloeckera, such as Kloeckera apiculata. In further embodiments, the parental fungal cell may be selected from the group comprising Schizosaccharomycetaceae, such as Schizosaccharomyces, for example Schizosaccharomyces pombe, Schizosaccharomyces japonicus, Schizosaccharomyces octosporus and Schizosaccharomyces cryophilus, especially selected from the group comprising Schizosaccharomyces pombe, Schizosaccharomyces octosporus and Schizosaccharomyces cryophilus; or from the group of Dothideomycetes, such as Aureobasidium pullulans; or from the group of Dipodascaceae, such as Yarrowia lipolytica. Particularly preferred are Saccharomyces cerevisiae, Xanthophyllomyces sp., S. japonicus or Yarrowia lipolytica.

### Modification or Enhancement of Yeasts

Modification means any genome modification as compared to the parent yeast, for example heterologous gene expression.

The term genome modification refers to a difference between two genomes, especially wherein the difference was deliberately introduced. In one embodiment, the genome modification may comprise one or more of an insertion, a deletion and a substitution of nucleotides. In a preferred embodiment, the genome modification comprises the insertion of an exogenous gene. Modifications include recombinant gene technology, mutations or gene editing.

In one embodiment, the modified yeast cell may be configured to express the exogenous gene. In particular, the modified yeast cells may be configured to transcribe an exogenous gene into mRNA. In particular, the modified yeast cells are configured to translate an exogenous mRNA into an exogenous protein.

In one embodiment, one or more exogenous genes encode a protein having squalenetetrahymanol cyclase activity and a protein having squalene-hopene cyclase activity. The activity may be determined by comparing the expression pattern of the modified cell as compared to a cell devoid of such a modification.

Enhancement means any improvement of the expression of an invertebrate nutrient as compared to the parent microorganism which has not been enhanced for the expression of this invertebrate nutrient, in particular cholesterol, desmosterol, 24-methylene cholesterol, and isofucosterol.

In one embodiment, the yeasts have been further modified or selected to produce an invertebrate nutrient selected from the group consisting of cholesterol, desmosterol, 24-methylene cholesterol, and isofucosterol.

In one embodiment, the yeasts have been modified or enhanced as compared to a parent yeast to express a squalene-hopene cyclase enzyme or a squalene-tetrahymenol cyclase enzyme to enable growth under anoxic conditions.

### Enhanced or Modified Yeast Cells

In one embodiment, cells of Saccharomyces cerevisiae, Xanthophyllomyces sp., S. japonicus or Yarrowia lipolytica are administered to invertebrates, in particular to honey bees and to bumble bees, wherein the yeast cells have been enhanced or modified to express or produce an invertebrate nutrient selected from the group consisting of cholesterol, desmosterol, 24-methylene cholesterol, and isofucosterol.

### Squalene-Hopene Cyclase or a Squalene-Tetrahymenol Cyclase

In one embodiment, the yeast has been modified to express a squalene-hopene cyclase enzyme or a squalene-tetrahymenol cyclase enzyme in order to allow it to grow in anoxic conditions. Preferably, no sterols or a reduced amount of sterols need to be added to the culture medium of the modified yeast as compared to a parent yeast cell that lacks this modification. WO 2021/133171 to the University of Delft provides examples of such modified yeast cells and methods of modifying yeast cells to express squalene-hopene cyclase or a squalene-tetrahymenol cyclase. A preferred example is example 4 of WO 2021/133171 to the University of Delft.

### Anoxic or Aerobic Culture Conditions

In one embodiment, the modified or enhanced microorganisms is S. japonicus cultured under anoxic conditions. In such conditions, typically no sterols are formed due to the lack of oxygen. In this embodiment, the modified or enhanced microorganisms are engineered to produce non-ergosterols such as 24-methylene cholesterol, isofucosterol, cholesterol or desmosterol under aerobic conditions.

In another embodiment, the modified or enhanced microorganisms are species of yeast in which a squalene-hopene cyclase enzyme is expressed and/or squalene-tetrahymenol cyclase enzyme is expressed enabling growth under anoxic conditions and complementation of sterol function by hopenes. These are conditions under which no sterols can be formed due to lack of oxygen and the sterol function for the cell is complemented by hopene in both anoxic and aerobic conditions but in which engineered non-ergosterols, such as 24-methylene cholesterol, isofucosterol, cholesterol or desmosterol are formed under aerobic conditions.

In another embodiment, the modified or enhanced microorganisms are a species of yeast such as S. cerevisiae or Yarrowia in which a squalene-hopene cyclase enzyme is expressed and/squalene-tetrahymenol cyclase enzyme is expressed enabling yeast growth under anoxic conditions and the sterol function for the cell is complemented by hopene in both anoxic and aerobic conditions. In anoxic conditions, normally no sterols can be formed due to lack of oxygen. In this embodiment, the microorganisms have been enhanced or modified to produce non-ergosterols, such as 24-methylene cholesterol, isofucosterol, cholesterol or desmosterol under aerobic conditions, preferably without producing or expressing ergosterol.

### Aerobic Culturing Condition

In another embodiment, the modified or enhanced microorganisms are a species of yeast such as S. cerevisiae or Yarrowia, which are cultured under aerobic conditions to express or produce non-ergosterols, such as 24-methylene cholesterol, isofucosterol, cholesterol or desmosterol, preferably without producing or expressing ergosterol.

### Invertebrate Antinutrients

Yeasts such as Saccharomyces cerevisiae and Yarrowia lypolitica are some of the most important micro-organisms used for the commercial production of fats and other fat derived compounds such as sterols and steroids. Many marine algae and diatoms that contain a diverse array of sterols are also being engineered to produce lipid compounds (Rampan et al 2010, Gallo et al. 2020). Furthermore, several plant species have been engineered to produce or express specific sterols or lipid compounds (Sawai et al. 2014). Invertebrates have not been widely manipulated for lipid or sterol content, but have the potential to be in the future as information about their genomes and metabolic pathways are revealed. Most of these sources contain very low levels of sterols and as a whole could not be incorporated in bee food without concentrating or extracting the sterols which is cost-prohibitive.

Some fungi including S. cerevisiae Torula can be incorporated in bee food as a source of proteins, vitamins, and amino acids. The yeast used today in the food and feed industry have products fully based on yeast. However, these particular yeasts contain antinutrients for bees. In particular, the native sterols in most yeasts are ergosterol and zymosterol.

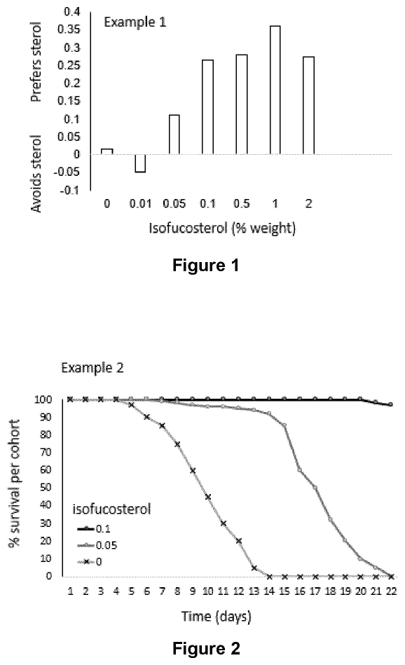

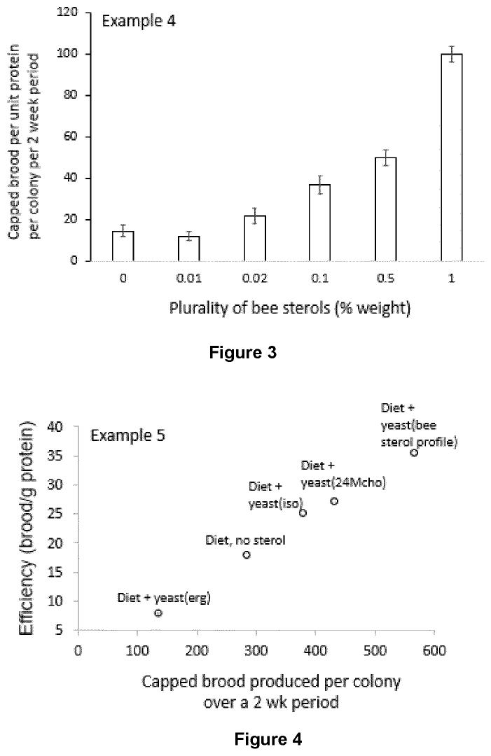

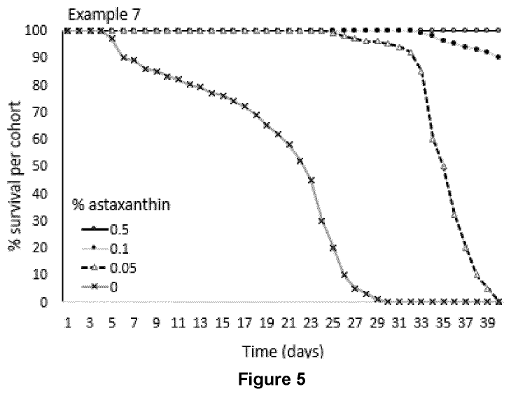

We have discovered that yeast containing ergosterol or ergosterol itself is not a preferred source of bee nutrition since bees actively avoid feed containing ergosterol in preference to food without it (FIG. 1). Bees have shorter lifespans when fed diets containing ergosterol (FIG. 2). Bees produce less brood on bee food with ergosterol than bee food that does not contain ergosterol (FIGS. 3 and 4). Ergosterol in bee diet does not cross the bee gut and is not metabolically useful. Instead, we have discovered that supplementation of the diet with ergosterol increases the Nosema population in the gut, resulting in intestinal microbiome dysbiosis and higher mortality (FIG. 5). Ergosterol supplementation is likely to benefit Nosema sp. (especially Nosema ceranae), as it is also used as a sterol by this pathogen. Ergosterol supplementation is also likely to increase gut populations of trypanosomes such as Crithidia bombi which also use ergosterol and that afflict bee species in the genus Bombus.

Thus, the inventors have discovered here that ergosterol and zymosterol, sterols common to many yeast species, are antinutrients for certain invertebrates including honey bees and bumble bees that harbour microsporidian or trypanosomal parasites. Yeasts, however, are beneficial sources of other nutrients for bees. Therefore, advantageously, we have engineered yeast species that provide other nutrients but which do not express these antinutrients in particular from the sterols found naturally in yeasts such as S. cereviseae. Advantageously, these genetically modified microorganisms or parts, extracts, oils or refinements thereof are delivered as part of an essentially pollen-free diet. This diet increases overall invertebrate performance, in particular bee brood production and survival of worker bees as compared to a non-pollen bee diet that does not contain such microorganisms.

Production of animals requires the development of cost-effective yet nutritionally optimal feeds. Commercially available bee feed compositions all contain yeast with ergosterol and zymosterol (as shown in Table 1). Consequently, the present invention is the first demonstration that the addition of yeast without ergosterol or zymosterol to a non-pollen bee diet is advantageous.

Engineering yeast strains to express or not specific compounds including sterols is a routine practice. Incorporating a yeast strain in nutrition that does not contain ergosterol or zymosterol or a mixture of these two sterols in nutrition is an inventive step.

The principle for making such yeast strains is well known to those experienced in the art:
An ergosterol deficient strain of S. cerevisiae can for example be made by deleting the genes erg4 and erg5 or erg5 alone in S. cerevisiae (eg. BG4742 erg4 erg5, Sawai et al. 2014). The same can be done in Yarrowia lipolytica by deleting the orthologues of erg4 and erg5.
An ergosterol-deficient strain of any other yeast species can for example be made by deleting the orthologs for the genes erg4 and erg5 in that species (eg. BG4742 erg4 erg5 in S. cerevisiae, Sawai et al. 2014).
An ergosterol deficient strain of S. cerevisiae can for example be made by deleting the genes erg4 and erg5 or erg5 alone in S. cerevisiae (eg. BG4742 erg4 erg5, Sawai et al. 2014). The same can be done in Yarrowia lipolytica by deleting the orthologues of erg4 and erg5). Such ergosterol-deficient strain can be made to express another sterol useful to bees such as campesterol as its primary (>90%) sterol by expressing the DWF1 cDNA or DHCR7 gene any enzyme with the same activity in this strain (Strain T55).

TABLE 1
Commercial pollen substitutes contain ergosterol and zymosterol from yeast

| microg/mg | Royal Jelly | Yeast | Ultra B | Mega B | AP23 | BPro | Brdbldr | Feed B | Asgogen |
|---|---|---|---|---|---|---|---|---|---|
| Zymosterol | 0 | 0.021 | 0.023 | 0.007 | 0.014 | 0.030 | 0.029 | 0.005 | 0.604 |
| Ergosterol | 0 | 0.298 | 0.208 | 0.058 | 0.192 | 0.277 | 0.289 | 0.052 | 1.530 |
| Total anti- | 0 | 0.319 | 0.231 | 0.065 | 0.206 | 0.307 | 0.318 | 0.057 | 2.133 |
| nutrients | | | | | | | | | |

Non-Pollen Composition The composition of the invention is advantageously a non-pollen composition.

The feature “non-pollen” means essentially free of pollen. However, minor amounts of pollen may be present in the compositions of the present inventions. In one embodiment, the amount of pollen is 15 w % or less, preferably 10 w % or less, even more preferably 5 w % or less and even more preferably 1 w % or less and even more preferably 0.1 w % or less as compared to the dry weight of the composition.

In another embodiment, the composition is a pollen substitute composition that replaces a pollen diet.

### Invertebrates

Invertebrate species are the most diverse animals on earth. They are important components of natural ecological systems and play key roles in the production of human food. An increasingly important area of food production involves the cultivation of invertebrates such as insects, crustaceans, and molluscs as food for humans and for livestock and for their roles in ecosystem services such as pollination. Insects are also cultivated as natural enemies of insect pests and released as part of integrated pest management strategies.

Invertebrates include
arthropods, such as insects, arachnids, crustaceans, and myriapods,
molluscs, such as chitons, snail, bivalves, squids, and octopuses,
annelid, such as earthworms and leeches; and
cnidarians, such as hydras, jellyfishes, sea anemones, and corals.

Preferred invertebrates are invertebrates that are cultured or farmed for purposes of human or animal nutrition such as bees, bumble bees, earthworms, meal worms, shrimps, prawns or crayfish, crickets and fly larvae. Particularly preferred invertebrates are those of the Apidae family which are used as pollinators for agricultural or horticultural plants, such as
bees of the genus Apis and in particular Apis mellifera or
bumble bees of the genus Bombus and in particular Bombus terrestris

### Ergosterol-Free

The composition of the invention does not contain or comprise, i.e. is essentially free of ergosterol, zymosterol or combinations thereof.

However, minor amounts of ergosterol may be present in the compositions of the present inventions. In one embodiment, the amount of ergosterol, zymosterol or combinations thereof is 5 w % or less, preferably 1 w % or less, even more preferably 0.1 w % or less as compared to the dry weight of the composition.

### Dosage and Concentration

The ergosterol/zymosterol-free yeast must be administered in an amount that is nutritionally effective for bees.

In one embodiment, nutritionally effective means the amount of ergosterol/zymosterol—free yeast in an amount as part of a pollen substitute composition, wherein the pollen substitute composition comprises:
proteins in an amount from 10 w % to 50 w %, preferably of 20 w % to 40 w %,
fatty acids in an amount from 1 w % to 20 w %, preferably of 2 w % to 12 w %,
carbohydrates in an amount from 30 w % to 90 w %, preferably of 5 w % to 50 w %,
optionally vitamins, and
optionally minerals,
   wherein the total amount of components and optionally further components add up to 100 w % and wherein the w % are related to the total dry weight of the composition.

In another aspect, the ergosterol/zymosterol-free yeast or a mixture thereof is mixed with sucrose and/or invert syrup in an embodiment of 1-60% yeast, but preferably 10-50% yeast, and more preferably 15-30% yeast.

### EXAMPLES

### Example 1. Preference Assay: Bees Avoid Ergosterol in Foods

Method: Newly emerged adult worker honey bees (Apis mellifera) or adult worker bumble bees (Bombus terrestris) were tested in a two-choice preference assay in which bees had access to two diets and adlibitum access to water. One treatment diet contained the ergosterol and the other contained no sterol. Newly emerged bees were removed from the brood frame and cohorts of 30 bees per replicate were housed in plastic rearing cages. In all experiments, 10 cohorts of ˜30 bees each were used for each treatment group. In all diets carbohydrate was maintained at 60% using powdered sugar and fat was maintained at 8% using an emulsion. Consumption of each diet was measured every 24 h for 5 days. Preference index was calculated as (amount of treatment consumed-amount of control consumed)/(total amount of food consumed).

### Example 2: Survival: Bees are Less Likely to Survive on Diets Containing Ergosterol

Method: Newly emerged adult worker honey bees fed with a diet treatment and ad libitum access to water. The treatment diet contained the ergosterol. Newly-emerged bees were removed from the brood frame and cohorts of 30 bees per replicate were housed in plastic rearing cages. In all experiments, 10 cohorts of ˜30 bees each were used for each treatment group. In all diets carbohydrate was maintained at 60% using powdered sugar and fat was maintained at 8% using an emulsion. Consumption of each diet was measured every day over the course of the experiment. The number of bees alive in the box was counted each day for 14 days.

Example 3 and 4. Brood Production: Honey Bees Cannot Produce Brood on Diets with high quantities of ergosterol Method: Fully functional insulated styrofoam APIDEA nucs comprised 5 mini frames populated with adult workers and 1 mated laying queen bee were populated with 300-400 ml of young adult workers (˜N<1,000 bees of mixed ages). The colony was located in an enclosed glasshouse with ventilation which did not permit the honey bees to forage on nectar or pollen. Each treatment was tested with 3-6 colonies; each colony was fed with a 60-100 g patty (solid diet) on the top feeder fitted with a mesh floor. Diet was fed on the first day and again on day 6; the quantity consumed was measured on day 6 and day 15. If no larvae or eggs/queen are observed by day 6 then the experiment is terminated. The number of capped brood cells was counted every 15 days. The number of bee seams was estimated during each inspection. Sugar syrup (34%) and water was provided in feeders inside the tent to prevent carbohydrate starvation and to stimulate foraging activity.

### Example 5. Nosema Infection: Honey Bees have Significantly Greater Nosema Loads when Fed with Ergosterol-Containing Diets

Method: Newly emerged adult worker honey bees (Apis mellifera) or adult worker bumble bees (Bombus terrestris) were reared in an assay to test whether diet induced an increase in Nosema ceranae infection. Bees had access to a diet composed of 15% protein, 6% fat, 78% carbohydrates and 1% vitamins and minerals and adlibitum access to water and sucrose solution. One treatment diet contained the ergosterol and the other contained no sterol. Newly emerged bees were removed from the brood frame and cohorts of 30 bees per replicate were housed in plastic rearing cages. In all experiments, 10 cohorts of ˜30 bees each were used for each treatment group. Consumption of each diet was measured every 24 h for 14 days.

Nosema ceranae infection of each cohort was produced as in Tischler et al. (2017). Briefly, Nosema spores were collected in 10 bees, concentrated and cultured. Cultures were added to 30% sucrose solutions and fed to the bees.

The following replaces all claims:

1. A method for feeding invertebrates, comprising
Providing a pollen substitute or supplement composition for invertebrates, in particular for honey bees and bumble bees out of yeast selected from the group consisting of S. cerevisiae, Xanthophyllomyces sp., S. japonicus or Yarrowia lipolytica,
Administering the composition to invertebrates,
wherein the composition is essentially free of ergosterol or zymosterol or combinations thereof.

2. The method of claim 1, wherein the yeast has been modified or enhanced not to express ergosterol or zymosterol.

3. claim 1, wherein the yeast has been modified or enhanced as compared to a parent yeast to express a squalene-hopene cyclase enzyme or a squalene-tetrahymenol cyclase enzyme to enable growth under anoxic conditions.

4. claim 1, wherein the yeast has been further modified to express an invertebrate nutrient selected from the group consisting of cholesterol, desmosterol, 24-methylene cholesterol.

5. claim 1, wherein the invertebrate nutrient is produced under aerobic conditions.

6. claim 1, wherein the sterol that is provided to or produced by the yeast for growth in lieu of ergosterol or zymosterol is not an antinutrient for bees.

7. claim 1, wherein a pollen substitute or supplement composition is created for honey bees and bumble bees that does not contain more than 0.01 microg/mg zymosterol.

8. claim 1, wherein a pollen substitute or supplement composition is provided for honey bees and bumble bees that does not contain more than 0.01 microg/mg ergosterol.

9. claim 1, wherein a pollen substitute or supplement is provided for honey bees and bumble bees that does not contain more than 0.01 microg/mg total of any one of the following sterols: ergosterol or zymosterol or their combinations.

10. claim 1, wherein the composition comprises:
proteins in an amount from 10 w % to 50 w %, preferably of 20 w % to 40 w %,
fatty acids in an amount from 1 w % to 20 w %, preferably of 2 w % to 12 w %,
carbohydrates in an amount from 30 w % to 90 w %, preferably of 50 w % to 70 w %,
wherein the total amount of components add up to 100 w % and wherein the w % are related to the total dry weight of the composition.

11. Use of the pollen substitute composition according to claim 1 for feeding invertebrates, in particular honey bees or bumble bees.
